(19) United States
(12) Patent Application Publication
(10) Pub. No.: US 2003/0171773 A1
(43) Pub. Date: Sep. 11, 2003

(54) Methods for aneurysm repair

(76) Inventor: Harold F. Carrison, Pleasanton, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/093,264

(22) Filed: Mar. 6, 2002

(51) Int. Cl. A61B 17/08

(52) U.S. Cl. 606/213

(57) ABSTRACT

Disclosed herein are methods of occluding a selected target site using rapidly expanding materials.

### FIELD OF THE INVENTION

Methods for the repair of aneurysms are described. In particular, use of foam or liquid material that rapidly expands upon extrusion from a delivery device to occlude selected sites are described.

### BACKGROUND

An aneurysm is a dilation of a blood vessel (similar to a balloon) that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings which may be dimensioned to engage the walls of the vessels. (See, e.g., U.S. Pat. No. 4,994,069 to Ritchart et al.) Other less stiff helically coiled devices have been described, as well as those involving woven braids.

Liquid embolics, such as cyanoacrylate glues and fibrin sealants, have also been used in animal and human subjects. See, e.g., Interventional Radiology, Dandlinger et al, ed., Thieme, N.Y., 1990:295-313; Suga et al. (1992) No Shinkei Geka 20(8):865-873; Moringlane et al. (1987) Surg Neurol 28(5):361-366; Moringlane et al. (1988) Acta Neurochir Suppl (Wein) 43:193-197. Of these liquid embolics, only cyanoacrylate glues are currently available to neurosurgeons. However, chronic inflammation is typically seen with cyanoacrylate treatments (Herrera et al. (1999) Neurol Med Chir (Tokyo) 39(2): 134-139) and the degradation product, formaldehyde, is highly toxic to the neighboring tissues. See, Vinters et al (1995) Neuroradiology 27:279-291. Another disadvantage of cyanoacrylate materials is that the polymer will adhere both to the blood vessel and to the tip of the catheter. Thus physicians must retract the catheter immediately after injection of the cyanoacrylate embolic material or risk adhesion of the cyanoacrylate and the catheter to the vessel.

None of these documents describe use of a material that rapidly expands upon extrusion (with no further expansion over time) for treating and occluding aneurysms.

### SUMMARY OF THE INVENTION

Thus, this invention includes novel methods of occluding a selected vessel using rapidly expanding materials not previously used for these purposes.

In one aspect, the invention includes a method of occluding a selected site in a subject comprising accessing the selected site; and administering a rapidly expanding composition to said selected site, thereby occluding said site. In certain embodiments, the composition comprises at least one foam, for example polyurethane or a dehydrated foam (e.g., a dehydrated foam comprising collagen). In other embodiments, the composition comprises two or more materials that rapidly expand upon contact with each other. In yet other embodiments, any of the compositions for use in the methods described herein are rapidly hardening.

In any of the methods described herein, the selected site is an aneurysm and the administering comprises extrusion from a catheter. Further, in any of the methods described herein, the rapidly expanding composition may self-expand; expand upon contact with moisture; expand upon a decrease in pressure (e.g., upon extrusion from a delivery device such as a catheter); and/or expand upon contact with an additional material (e.g., where the contact is at the tip of the catheter).

In other aspects, any of the methods described herein further comprise the step of administering one or more bioactive materials (e.g., cytokines; trace metals, antibiotics and combinations thereof), either before, after or concurrently with the composition.

These and other embodiments of the subject invention will readily occur to those of skill in the art in light of the disclosure herein.

### DESCRIPTION OF THE INVENTION

This invention involves a methods of occluding a selected vessel in a subject using a material (e.g., liquid or foam) that rapidly expands upon extrusion from a deployment mechanism and does not further expand over time. The material may include additives and/or fillers (such as radio-opaque additives and/or bioactive materials such as cytokines, growth factors, etc.) or the like. As used herein the term “expansion” refers to any increase in volume. The term “rapidly expanding” refers to any material that expands upon, for example extrusion, within a short period of time (on the order of seconds or less than a second) and does not further expand after this initial expansion over time. Similarly, the term “hardening” or “solidifying” refers to any liquid or flowable material that forms a solid mass with a particular expanded volume, either over time, upon contact with another substance or upon application of energy. Furthermore, the term “rapidly hardening” refers to any of these materials that solidify in less than about one hour, preferably less than 5 minutes, and even more preferably, less than about 30 seconds. Thus, the rapidly expanding materials described herein are to be contrasted with materials that take on the order of hours or days to stop expanding and form clots with desirable strength.

The methods described herein are suitable for both neurological, peripheral and cardiovascular applications as well as in other vessels such as in the Fallopian tubes and the like.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise. Thus, for example, reference to “a rapidly expanding material” includes a mixture of two or more such materials and the like.

Any suitable rapidly expanding material can be used, so long as it is not harmful when implanted in the subject. The material will preferably have certain characteristics: (1) it should be flowable (e.g., liquid, paste or foam) at delivery; (2) it should expand upon extrusion from the deployment mechanism (e.g., catheter); (3) expansion upon extrusion should be immediate or virtually immediate, on the order to seconds or less than a second; (4) no expansion should occur after the initial expansion on extrusion; (5) hardening is preferably rapid, seconds or minutes rather than hours or days; (6) few or no toxic or otherwise harmful by-products should be released; and (7) the resultant expanded material must not significantly expand its volume after initial expansion.

Thus, non-limiting examples of potentially suitable materials include liquids, pastes, foams and other flowable materials. In certain aspects, the rapidly expanding material is a single component material. Typically, single component materials are those that rapidly expand when released from a pressurized system or, alternatively, upon contact with moisture. Pressurized fluids expand upon exiting the neck of the catheter as they go from higher to lower pressure. Pressure differentials can range anywhere between 10 to 1,000 or more PSI. For example, the material sold under the name Silly String™ expands upon release from a pressurized contained, and, after the initial expansion upon extrusion, does not further expand over time. Preferably, the material also hardens virtually instantaneously upon extrusion.

In other embodiments, multi-component materials are used, so long as the expansion that occurs rapidly at the site of deployment does not continue over time. Other examples of single component materials include those materials that rapidly expand upon contact with moisture, for example certain polyurethane foam materials (e.g., U.S. Pat. No. 4,2724,250; and Lu et al. (2000) BioMaterials 21(15):1595-1605 describing porous poly(L-lactide acid foams); hydrophilic polymers as disclosed, for example, in U.S. Pat. No. 5,162,430; hydrogel materials such as those described in Wake et al. (1995) Cell Transplantation 4(3):275-279, Wiese et al. (2001) J. Biomedical Materials Research 54(2):179-188 and Marler et al. (2000) Plastic Reconstruct. Surgery 105(6):2049-2058; hyaluronic acid materials (e.g., Duranti et al. (1998) Dermatologic Surgery 24(12):1317-1325); and expanding beads such as chitin beads (e.g., Yusof et al. (2001) J. Biomedical Materials Research 54(1):59-68). A non-limiting example of material that rapidly expands upon contact with moisture is a dehydrated foam, for example a dehydrated foam containing collagen.

Non-limiting examples of multi-component materials include calcium phosphate cements (see, e.g., U.S. Pat. No. 6,159,655) made by mixing two more components into a flowable paste which then subsequently expands and/or hardens and mixtures of any of any suitable single-component materials. Additionally, one or more of materials can also be used in various combinations.

As noted above, the rapidly expanding material is preferably in a fluid or foam state for delivery, for example for transport through the lumen of a catheter to the site to be occluded. Additional agents may be present to facilitate delivery of the rapidly expanding material, so long as the additional fluid is not harmful to the subject may be used. For example, the rapidly expanding material can be dispersed in a water-blowing agent (see, e.g., U.S. Pat. No. 6,211,257 and references cited therein).

The rapidly expanding materials can be used alone or in combination with one or more implantable devices (e.g., vaso-occlusive devices, stents, filters, etc.), one or more additional bioactive materials or a combination of implantable devices and additional bioactive materials. Suitable implantable devices are known to those skilled in the art. The term “bioactive” refers to any agent which exhibits effects in vivo, for example a thrombotic agent, a therapeutic agent or the like. Non-limiting examples of bioactive materials include cytokines; trace metals (e.g., copper); molecules that stabilize thrombus formation or inhibit clot lysis (e.g., proteins or functional fragments of proteins, including but not limited to Factor XIII, alpha₂-antiplasmin, plasminogen activator inhibitor-1 (PAI-1)); antibiotics; DMSO; or the like. Non-limiting examples of cytokines which may be used alone or in combination in the practice of the present invention include, basic fibroblast growth factor (beta-FGF), platelet derived growth factor (PDGF), vascular endothelial growth factor (VEGF), transforming growth factor beta (TGF-β) and the like. Cytokines are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). Additionally, bioactive polypeptides can be synthesized recombinantly as the sequence of many of these molecules are also available, for example, from the GenBank database. It is intended, although not always explicitly stated, that molecules having similar biological activity as wild-type or purified cytokines and thrombus-stabilizing proteins (e.g., recombinantly produced or mutants thereof) and nucleic acid encoding these molecules are intended to be used within the spirit and scope of the invention. Further, the amount and concentration of bioactive materials useful in the practice of the invention can be readily determined by a skilled operator and it will be understood that any combination of materials, concentration or dosage can be used, so long as it is not harmful to the subject.

Conventional catheter insertion and navigational techniques involving guidewires or flow-directed devices may be used to access the site to be occluded. The mechanism will be such as to be capable of being advanced entirely through the catheter to place implantable device at the target site but yet with a sufficient portion of the distal end of the delivery mechanism protruding from the distal end of the catheter to enable detachment of the implantable device. For use in peripheral or neural surgeries, the delivery mechanism will normally about 100-200 cm in length, more normally 130-180 cm in length. The diameter of the delivery mechanism is usually in the range of 0.25 to about 0.90 mm. Briefly, the rapidly expanding materials described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g., mechanical devices and/or rapidly expanding materials and bioactive materials) which cause formation of an emboli and, at some later time, is at least partially replaced by neovascularized collagenous material formed around the implanted devices.

A selected site is reached through the vascular system using a collection of specifically chosen catheters and/or guide wires. Suitable catheters known to those of skill in the art and include flexible catheters (see, e.g., U.S. Pat. Nos. 6,165,163; 6,159,187; 6,090,099; and 4,739,768) and multi-lumen catheters (e.g., U.S. Pat. Nos. 5,797,869 to Martin et al.; 4,636,346, to Gold et al.; 4,840,622, to Hardy; 4,863,442, to DeMello et al.; and 5,078,702, to Pomeranz). Catheters suitable for delivering materials which expand upon reduced pressure include those including pinch valves and/or other means for keeping a fluid under pressure within the confines of the catheter. (see, e.g., U.S. Pat. No. 6,210,319 and U.S. Pat. No. 6,210,392 and documents cited therein).

It is clear that should the selected site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter as described herein are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the rapidly expanding material is advanced through the catheter.

The rapidly expanding material and/or other materials is(are) advanced past the distal end of the catheter and positioned or extruded precisely at the desired treatment site where they rapidly expand to fill the target site. When used with other devices and/or materials, the order in which the components (e.g., rapidly expanding material; vaso-occlusive member; and/or other bioactive materials) are released from the catheter is not critical to the practice of the invention. Nonetheless, when used in conjunction with an implantable device, it may be preferable to release the rapidly expanding material after the device is situated so that the fluid can penetrate in and around the device.

Modifications of the procedures described above will be apparent to those having skill in this mechanical and surgical art. These variations are intended to be within the scope of the claims that follow.

What is claimed is:

1. A method of occluding a selected site in a subject comprising
accessing the selected site;
administering a rapidly expanding composition to the selected site, thereby occluding said site.

2. The method of claim 1, wherein the composition comprises one or more foams.

3. The method of claim 2, wherein the foam comprises polyurethane.

4. The method of claim 1, wherein the composition rapidly expands upon contact with moisture.

5. The method of claim 1, wherein the composition rapidly expands upon a decrease in pressure.

6. The method of claim 1, wherein the composition comprises two materials that rapidly expand upon contact with each other.

7. The method of claim 1, wherein the administering comprises extruding the composition from a catheter.

8. The method of claim 7, wherein the composition rapidly expands upon contact with an additional material and wherein said contact is at the tip of the catheter.

9. The method of claim 7, wherein the material rapidly expands in response to a decrease in pressure upon extrusion from the catheter.

10. The method of claim 1, further comprising administering one or more bioactive materials.

11. The method of claim 10, wherein the bioactive materials are administered prior to the composition.

12. The method of claim 10, wherein the bioactive materials are administered concurrently with the composition.

13. The method of claim 10, wherein the bioactive materials are administered subsequent to the composition.

14. The method of claim 10, wherein the bioactive materials are selected from the group consisting of cytokines; trace metals, antibiotics and combinations thereof.

15. The method of claim 1, wherein the selected site is an aneurysm.

16. The method of claim 1, wherein the composition is rapidly hardening.

17. The method of claim 4, wherein the composition comprises a dehydrated foam.

18. The method of claim 17, wherein the dehydrated foam comprises collagen.
